(19) United States
(12) Patent Application Publication
(10) Pub. No.: US 2022/0354608 A1
(43) Pub. Date: Nov. 10, 2022

(54) Product Retention Tray

(71) Applicant: Prent Corporation, Janesville, WI (US)

(72) Inventors: Donald L. Handrow, Janesville, WI (US); Jennifer A. Stingle, Janesville, WI (US)

(73) Assignee: Prent Corporation, Janesville, WI (US)

(21) Appl. No.: 17/661,941

(22) Filed: May 4, 2022

Related U.S. Application Data
(60) Provisional application No. 63/201,545, filed on May 4, 2021.

(51) Int. Cl. A61B 50/33; A61B 50/20

(52) U.S. Cl. CPC A61B 50/33; A61B 50/20

(57) ABSTRACT

A tray is provided that includes a tray body with a tray body cavity formed therein with a medical device compartment formed in the tray body cavity for receiving a medical device at least partially within, the tray further includes one or more retention pockets formed in the tray body and situated in the tray body cavity, and one or more device retainers respectively situated at least partially within and coupled to the one or more retention pockets, wherein the one or more device retainers are translatable between an open position to allow the medical device to be inserted into the medical device compartment and a closed position for securing the medical device in the medical device compartment, and wherein the one or more device retainers remain coupled respectively to the one or more retention pockets in both the closed position and open position.

### CROSS-REFERENCE

This application claims priority to and incorporates by reference herein U.S. Provisional Patent Application Ser. No. 63/201,545 filed on May 4, 2021.

### FIELD OF THE INVENTION

This invention relates generally to product packaging in the field of medical devices.

### BACKGROUND

Medical devices/equipment for use in a surgical setting are typically sterilized and sealed in a thermoformed tray/package by the manufacturer. Prior to use, the device is unsealed and removed from the tray in the surgical setting. All items brought into a surgical setting must be accounted for to ensure the safety of the patient, such items would include the device, the tray, and any loose components associated with the tray.

### SUMMARY OF THE INVENTION

In at least some embodiments, a tray for securing a medical device is provided that includes: a tray body with a tray body cavity formed therein; a medical device compartment formed in the tray body cavity, for receiving a medical device at least partially within; one or more retention pockets formed in the tray body and situated in the tray body cavity; and one or more device retainers respectively situated at least partially within and coupled to the one or more retention pockets, wherein the one or more device retainers are translatable between an open position to allow the medical device to be inserted into the medical device compartment and a closed position for securing the medical device in the medical device compartment, and wherein the one or more device retainers remain coupled respectively to the one or more retention pockets in both the closed position and open position.

Other embodiments, aspects, and features of the invention will be understood and appreciated upon a full reading of the detailed description and the claims that follow.

### BRIEF DESCRIPTION OF THE DRAWINGS

Embodiments of the invention are disclosed with reference to the accompanying drawings and are for illustrative purposes only. The invention is not limited in its application to the details of construction or the arrangement of the components illustrated in the drawings. The invention is capable of other embodiments or of being practiced or carried out in various other ways.

FIG. 1 is a perspective top view of an exemplary embodiment of a medical device tray.

FIG. 2 a perspective bottom view of the medical device tray of FIG. 1.

FIG. 3A is a top view of the medical device tray of FIG. 1 with device retainers removed from the tray body.

FIG. 3B is a perspective top view of the medical device tray of FIG. 1 with device retainers removed from the tray body.

FIG. 4 is a partial close-up sectional view taken along lines 4-4 of FIG. 3A.

FIG. 5 is a perspective view of an exemplary medical device.

FIG. 6 is a perspective top view of an exemplary first device retainer of FIG. 1.

FIG. 7 is a front view of the first device retainer of FIG. 6.

FIG. 8 is a bottom view of the first device retainer of FIG. 6.

FIG. 9 is a perspective top view of an exemplary second device retainer of FIG. 1.

FIG. 10 is a bottom perspective view of the second device retainer of FIG. 9.

FIG. 11 is a side view of the second device retainer of FIG. 9.

FIG. 12 is a perspective top view of an exemplary third device retainer of FIG. 1.

FIG. 13 is a bottom perspective view of the third device retainer of FIG. 12.

FIG. 14 is a side view of the third device retainer of FIG. 12.

FIG. 15 is a perspective top view of the medical device tray of FIG.1 with the medical device of FIG. 5 positioned therein and the device retainers in open positions.

FIG. 16 is a top view of the medical device tray of FIG.15 with the device retainers in partially closed positions.

FIG. 17 is a top view of the medical device tray of FIG.15 with the device retainers in fully closed positions.

### DETAILED DESCRIPTION

To protect a surgical patient and to ease the burden of medical staff, a medical device tray has been disclosed that provides one or more device retainers to securely hold a medical device in the tray and which remain coupled with the tray before and after removal of a medical device. In at least some embodiments, the device retainers are recessed in the tray to limit opportunities for unintentional decoupling from the tray.

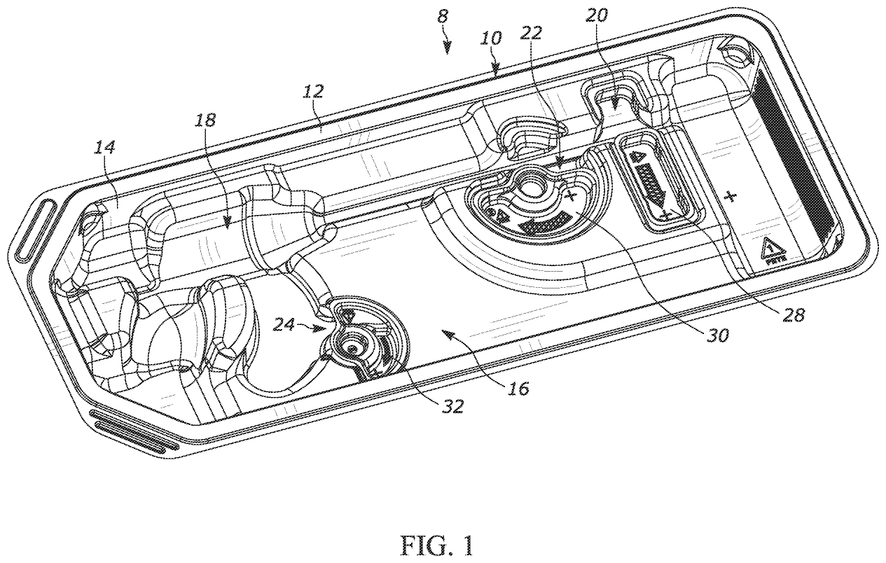

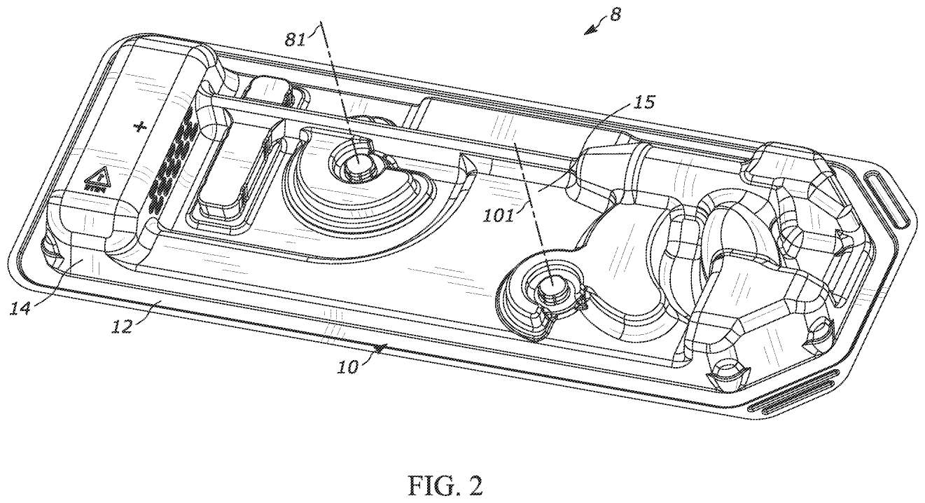

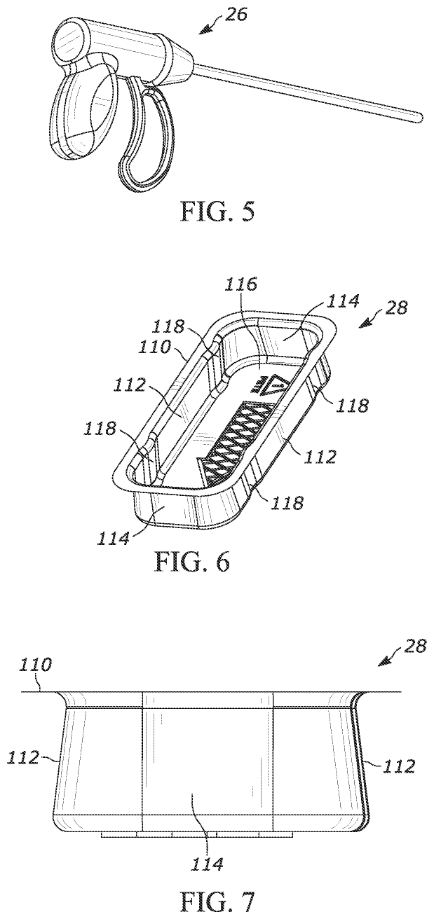

Referring to FIGS. 1 and 2, top and bottom perspective views of an exemplary embodiment of a medical device tray 8 are provided. The medical device tray 8 can take many forms and in at least some embodiments includes a tray body 10 with an outer perimeter tray flange 12 extending atop a plurality of walls 14 interconnected at least indirectly by a tray bottom 15, and forming at least in part, a tray body cavity 16 that further includes a medical device compartment 18 and one or more retention pockets, such as retention pockets 20, 22, 24. The device compartment 18 is sized and shaped to matingly receive at least a portion of a medical device 26 therein (see exemplary medical device in FIG. 5). The medical device 26 can include any number of types of medical devices, for example a Laparoscopic Trocar, or Endoscopic Stapler, etc.

One or more translatable device retainers, such as exemplary device retainers 28, 30, 32, are respectively coupled with the retention pockets. The referenced and illustrated retention pockets 20, 22, 24 and device retainers 28, 30, 32 are exemplary in shape, size, and position, and it shall be understood that the tray 8 can include various quantities including one or more of each to accommodate various types of medical devices.

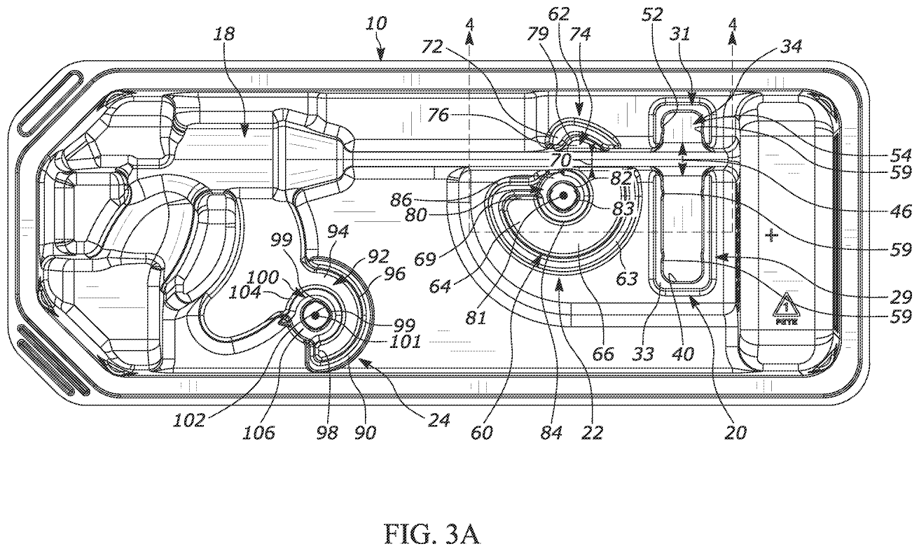

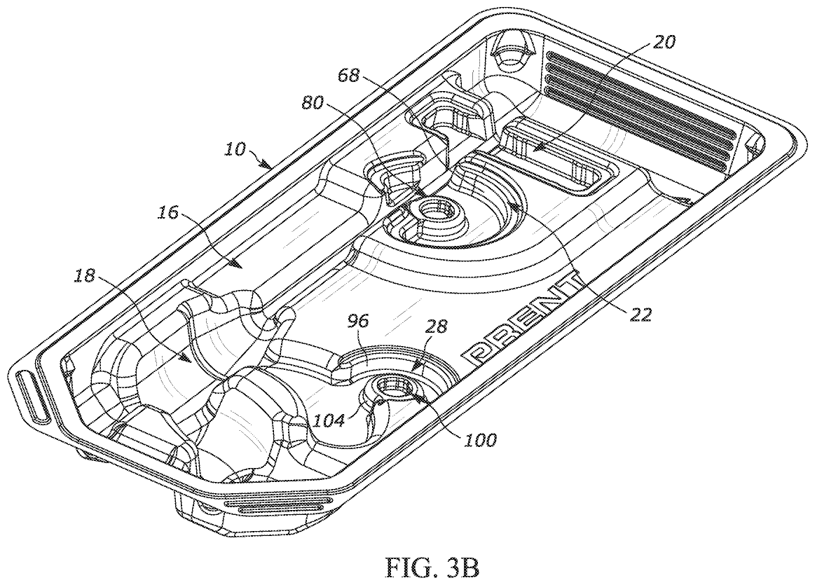

As best seen in FIG. 3A and 3B, illustrating a top and perspective view of the medical device tray of FIG. 1 with the device retainers removed for clarity, the retention pockets 20, 22, 24 are recessed relative to the tray flange 12 and are positioned adjacent to the device compartment 18. The retention pockets can take many forms and are configured to couple the device retainers to the tray body 10 while allowing the device retainers to be translatable between an open position for allowing the medical device to be received into the device compartment 18 and a closed position for securing the medical device in the device compartment 18.

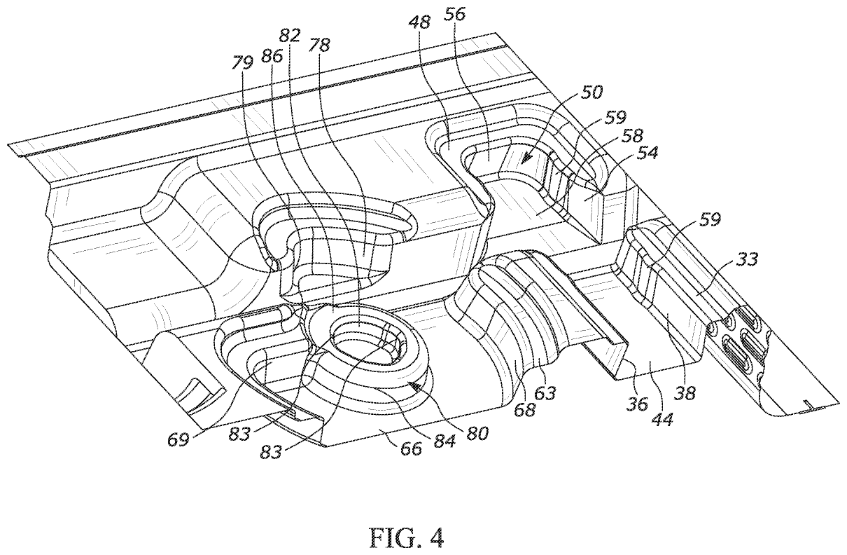

As medical devices come in various shapes and sizes, many variations of retention pockets can be utilized to allow for insertion and securement of the medical device. For example, as best seen in FIGS. 3A, 3B, and 4, retention pocket 20 can include a first portion 29 and a second portion 31. The first portion 29 includes a recessed ledge 33 at least partially surrounding a channel 34 having elongated retention side walls 36, 38 that are tapered, a retention end wall 40, and a generally flat channel bottom 44. Retention pocket 20 is configured to allow a portion of a medical device to pass lower than the channel bottom 44, meaning that the device compartment 18 extends below the depth of the channel bottom 44 and the device retainer 28 is longitudinally translatable to a closed position over a portion of the medical device securing the medical device between the device retainer 28 and the device compartment 18. The second portion 31 of the retention pocket 20 is spaced from the first portion 29 by a gap 46, wherein the medical device can pass through the gap 46. The second portion 31 is aligned with the first portion 29, and as such is configured similarly having a recessed ledge 48 at least partially surrounding a channel 50 with elongated tapered side walls 52, 54, a second end wall 56, and a generally flat channel bottom 58. Retention pocket 20 can also include one or more locking tabs 59 that protrude from the side walls 36, 38, 52, 54.

Retention pocket 22 is generally partially donut-shaped (e.g., curved, arced, etc.) and includes a first portion 60 and a second portion 62 (retention pocket 22 is also referred to as a first rotational retention pocket). Similar to retention pocket 20, the first portion 60 is separated from the second portion 62 by a gap 70 to allow for a medical device to pass therethrough. The first portion 60 includes a recessed ledge 63 at least partially surrounding an arced channel 64 having a channel bottom 66, a sidewall 68, and a first end wall 69. The second portion 62 is rotationally aligned with the first portion 60, and as such is configured similarly, having a recessed ledge 72 at least partially surrounding an arced channel 74 having a channel bottom 76, a sidewall 78, and a second end wall 79. The first portion 60 further includes a central ring 80 having a device retainer center axis 81 extending upwards from the plane of the channel bottom 66 and having an inner wall 82, an outer wall 84, and a ridge portion 86. The inner wall 82 is generally circular and can include an upwards taper and one or more locking recesses 83. The ring 80 is generally evenly spaced from the sidewall 68. The device retainer 30 is coupled to the ring 80 and is rotationally translatable from an open position to a closed position that extends over a portion of the medical device thereby securing the medical device between the device retainer 30 and the device compartment 18.

Retention pocket 24 is also generally partially donut-shaped (e.g., curved, arced, etc.) and includes a recessed ledge 90 at least partially surrounding an arced channel 92 having a channel bottom 94, a sidewall 96, and an end wall 98 (retention pocket 24 is also referred to as a second rotational retention pocket). A central ring 100 is provided, similar to the ring 80, that includes a device retainer center axis 101 extending upwards from the plane of the channel bottom 94 and having an inner wall 102, an outer wall 104, and a ridge portion 106. The inner wall 102 can include an upwards taper. In at least some embodiments, the central ring 100 is generally evenly spaced from the sidewall 96. The inner wall 102 can further include one or more locking recesses 99. The device retainer 32 is coupled to the central ring 100 and is rotationally translatable from an open position to a closed position that extends over a portion of the medical device to secure the medical device between the device retainer 32 and the device compartment 18.

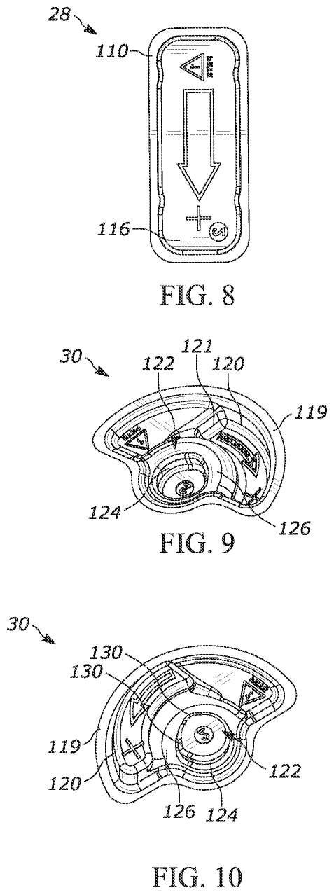

Referring now to FIGS. 6-8, the device retainer 28 is shown in top perspective, side, and bottom views. The device retainer 28 (also referred to as a slidable device retainer) is sized and shaped to generally matingly fit and slide within the retention pocket 20, and in at least some embodiments is generally tray-shaped. In at least some embodiments, the device retainer 28 includes a perimeter lip 110 surrounding the top of two retainer side walls 112, and two retainer end walls 114. In at least some embodiments, the side walls 112 and end walls 114 taper upwards from a bottom 116. The perimeter lip 110 is configured to ride along the recessed ledges 33, 48 of the retention pocket 20. One or more locking pockets 118 are provided along the elongated side walls 112 for mating engagement with the locking tabs 59 in the retention pocket 20. The device retainer 28 is coupled to tray body 10 by forcing the side walls 112 into the retention pocket 20 and in abutment with the side walls 36, 38, noting that the upwards taper of both the side walls 36, 38 of the retention pocket 20 and the side walls 112 of the device retainer 28 serve to retain the device retainer 28 within the retention pocket 20 (requiring notable force to remove), and the engagement of the perimeter lip 110 and ledges 33, 48 can also serve to create a positive sliding engagement to secure the device retainer 28 to the tray body 10, regardless of its position relative to the retention pocket 20.

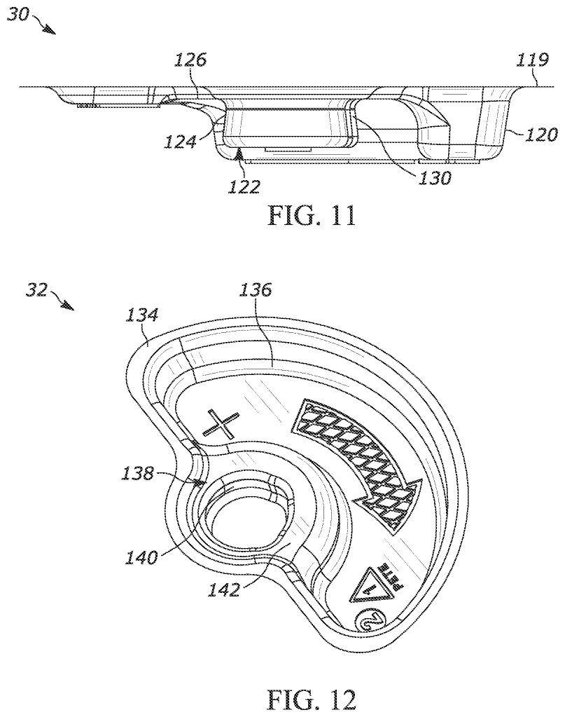

Referring now to FIGS. 9-11, the device retainer 30 is shown in top and bottom perspective, and side views. The device retainer 30 (also referred to as a first rotatable device retainer) is sized and shaped to generally matingly fit and rotate within the retention pocket 22, and in at least some embodiments is generally semi-circular/arc shaped. In at least some embodiments, the device retainer 30 includes a perimeter lip 119 extending around the top of the device retainer 30. The device retainer 30 includes a side wall 120 extending down from the perimeter lip 119, which in at least some embodiments tapers downward therefrom, along with an end wall 121. The device retainer 30 further includes a hub 122 having a generally circular hub wall 124 that, in at least some embodiments, tapers as it extends towards a top flange portion 126, wherein the top flange portion 126 is generally planar and is configured to rotate on the ridge portion 86 of the retention pocket 22. The device retainer 30 is coupled to retention pocket 22 by forcing the hub wall 124 into the ring 80 in abutment with the inner wall 82, noting that the upwards taper of both the inner wall 82 of ring 80 and the hub wall 124 along with the engagement of the top flange portion 126 on the ridge portion 86 serve to create a positive engagement to secure the device retainer 30 to the tray body 10 at a vertical point on the device retainer center axis 81 of the retention pocket 22, regardless of its rotatable position within the retention pocket 22. To assist with maintaining the device retainer in each of a closed and open position, the circular hub wall 124 can include one or more radially extending locking protrusions 130 positioned to matingly engage locking recesses 83 in the inner wall 82 of the ring 80 of retention pocket 22. The hub 122 is positioned to rotate about the device retainer center axis 81 of the retention pocket 22.

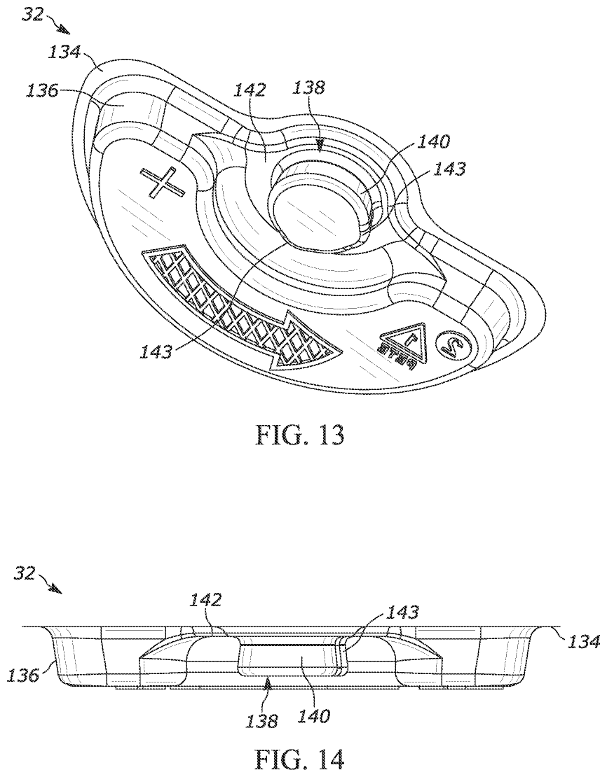

Referring now to FIGS. 12-14 the device retainer 32 is shown in top and bottom perspective, and side views. The device retainer 32 (also referred to as a second rotatable device retainer) is sized and shaped to generally matingly fit and rotate within the retention pocket 24 and in at least some embodiments is generally semi-circular/arc-shaped. In at least some embodiments, the device retainer 32 includes a perimeter lip 134 extending around the top of the device retainer 32. The device retainer 32 includes a side wall 136 extending down from the lip 134 and in at least some embodiments tapers downward therefrom. The device retainer 32 further includes a hub 138 having a generally circular hub wall 140 that in at least some embodiments tapers as it extends towards a top flange portion 142, wherein the top flange portion 142 is generally planar and is configured to rotate on the ridge portion 106 of the retention pocket 24. The device retainer 32 is coupled to retention pocket 24 by forcing the hub wall 140 into the central ring 100 in abutment with the inner wall 102 of central ring 100, noting that the upwards taper of both the inner wall 102 and the hub wall 124, along with the engagement of the top flange portion 142 on the ridge portion 106, serve to create a positive engagement to secure the device retainer 32 to the tray body 10 about a vertical point on the device retainer center axis 101 of the retention pocket 24, regardless of its rotatable position within the retention pocket 24. To assist with maintaining the device retainer 32 in each of a closed and open position, the circular hub wall 140 can include one or more radially extending locking protrusions 143 positioned to matingly engage locking recesses 99 in the inner wall 102 of the central ring 100 of retention pocket 24. The hub 138 is positioned to rotate about the device retainer center axis 101 of the retention pocket 24.

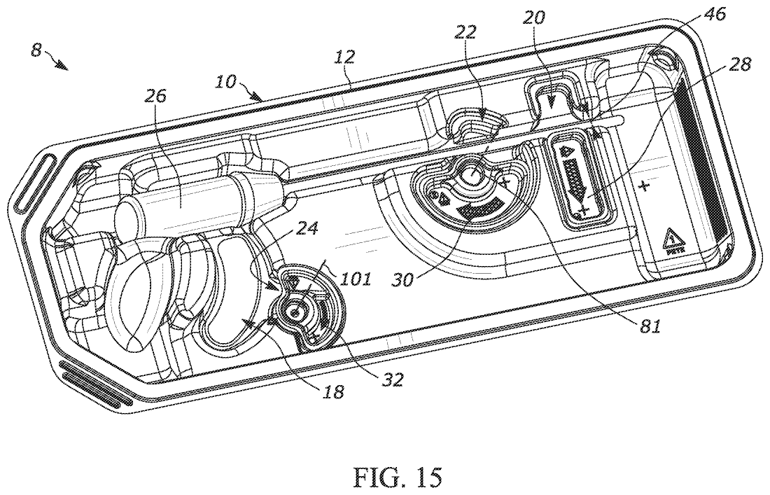

Referring now to FIGS. 15-17, the tray 8 is shown in various stages of securing the exemplary medical device 26 therein. In FIG. 15 the medical device 26 has been placed in the device compartment 18 of the tray body 10. The device compartment 18 is shaped and sized to receive the medical device 26 such that one or more portions of the medical device 26 pass through the gaps (e.g., 46, 70) of the retention pockets or are otherwise positioned below one or more device retainers (e.g., retainers 28, 30, 32) such that translation of the device retainers serves to releasably secure the medical device 26 to the tray body 10.

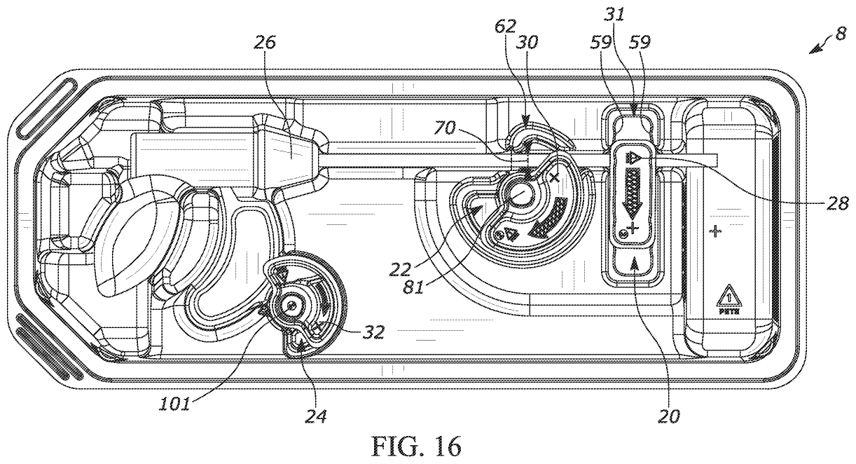

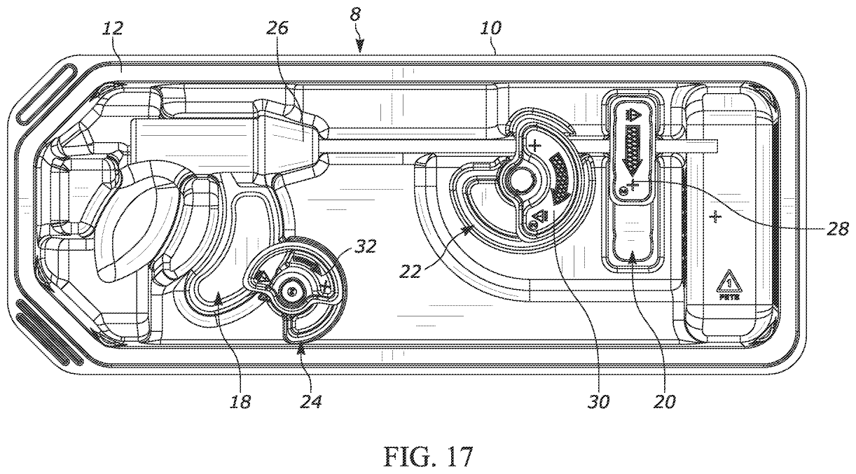

A seen in FIG. 16, device retainer 28 has been moved longitudinally towards the second portion 31 to a partially closed position to cover the gap 46 (see FIG. 15) that received the medical device 26 therethrough; and in FIG. 17, the device retainer 28 is shown in its fully closed position, where the device retainer 28 has been further moved to place the device retainer end wall 114 adjacent to or abutment with the end wall 56 of the second portion 31 of the retention pocket 20, further noting that the one or more locking tabs 59 of the retention pocket 20 engage the one or more locking pockets 118 of the device retainer 28 to provide a mating engagement therewith that secures the device retainer 28 in this closed position.

Referring again to FIG. 16, device retainer 30 has been rotated about the device retainer center axis 81 of the retention pocket 22 towards the second portion 62 to a partially closed position to cover the gap 70 that received the medical device 26 therethrough; and in FIG. 17 showing the device retainer 30 in its fully closed position, it can be seen that device retainer 30 has been rotated to place the device retainer end wall 121 adjacent to or abutment with the end wall 79 of the second portion 62 of the retention pocket 22, further noting that the one or more radially extending locking protrusions 130 along the circular hub wall 124 engage the locking recesses 83 in the inner wall 82 of the ring 80 of retention pocket 22 to provide a mating engagement therewith to limit or prevent rotation of the device retainer 30 back to the open position and securing the device retainer 30 in this closed position.

Referring further to FIG. 16, device retainer 32 has been rotated about the device retainer center axis 101 of the retention pocket 24 to a partially closed position to cover a portion of the medical device 26; and in FIG. 17 showing the device retainer 32 in its fully closed position, it can be seen that the device retainer 32 has been further rotated until the one or more of the radially extending locking protrusions 143 along the circular hub wall 140 engage one or more locking recesses 99 in the inner wall 102 of the central ring 100 of retention pocket 24 to provide a mating engagement therewith to limit or prevent rotation of the device retainer 32 out of the closed position.

In at least some embodiments, variations in the tray body 10 and device retainers 30, 32 can include alternate configurations for providing locking features to assist with maintaining the device retainers in a closed and/or open position. For example, inward extending locking protrusions can be provided along the side walls 68, 96 of the rotational retention pockets, which matingly engage locking recesses formed in the device retainers side walls 120, 136. Further, in at least some embodiments, variations in the retention pocket 20 can include a dog-bone shape such that the channel 34 has a narrower portion that is formed by the retention side wall 38 and recessed ledge 33 being shifted inward as they extend to the gap 46, to improve loading of the slidable device retainer 28.

Although not shown, once the medical device 26 has been secured in the tray body 10, the tray 8 can be sealed, such as with a plastic covering adhered to the tray flange 12 or wrapped completely around the tray body 10. When the tray 8 has been brought into a surgical setting where the medical device 26 is to be used, the wrapping can be removed and the device retainers can be moved back to their open positions to allow the medical device to be removed. Due to the engagement features discussed above, the device retainers remain secured to the tray body 10, whether in a closed or open position, thereby allowing the tray 8 to be counted as a single item in surgical setting.

Various aspects of the tray can be modified within the spirit of the invention. In addition to the disclosed shapes and sizes, all the aforementioned components, can vary to include numerous adaptations. Further, the material composition of all components can include numerous materials, such as plastics, metals, etc. In at least some embodiments, the tray is thermoformed, while in other embodiments other methods for forming can be utilized. The use of the term “plurality” in the description or claims shall be understood to include “one or more.”

While the invention is particularly useful for use with medical devices, other applications are possible and references to use with medical devices should not be deemed to limit the application of the invention. Rather, the invention may be advantageously adapted for use where similar performance capabilities and characteristics are desired, and that such modifications may be made by one of skill in the art of the invention without departing from the spirit or intent of the invention and therefore, the invention is to be taken as including all reasonable equivalents to the subject matter.

What is claimed is:

1. A tray for securing a medical device comprising:
a tray body with a tray body cavity formed therein;
a medical device compartment formed in the tray body cavity, for receiving a medical device at least partially within;
one or more retention pockets formed in the tray body and situated in the tray body cavity; and
one or more device retainers respectively situated at least partially within and coupled to the one or more retention pockets, wherein the one or more device retainers are translatable between an open position to allow the medical device to be inserted into the medical device compartment and a closed position for securing the medical device in the medical device compartment, and wherein the one or more device retainers remain coupled respectively to the one or more retention pockets in both the closed position and open position.

2. The tray of claim 1, wherein the one or more device retainers are at least one of rotatable and longitudinally slidable between the closed position and the open position.

3. The tray of claim 1, wherein the one or more device retainers is a slidable device retainer that is longitudinally slidable between the closed position and the open position within the one or more retention pockets.

4. The tray of claim 3, wherein the slidable device retainer includes two elongated retainer side walls and two retainer end walls that extend from a bottom to a perimeter lip.

5. The tray of claim 4, wherein the two elongated retainer side walls and one or more retainer end walls of the slidable device retainer taper as they extend upwards from the bottom towards the perimeter lip.

6. The tray of claim 5, wherein at least one of the one or more retention pockets is configured to matingly receive the slidable device retainer and further comprises a first portion and a second portion, wherein the first portion includes a first recessed ledge at least partially surrounding a channel having elongated first retention side walls that are tapered, a first retention end wall, and a substantially flat first retention channel bottom, and the second portion is spaced from the first portion by a gap, the second portion being aligned with the first portion, and having a second recessed ledge at least partially surrounding a second channel with elongated tapered second retention side walls, a second retention end wall, and a substantially flat second retention channel bottom.

7. The tray of claim 6, wherein translation of the slidable device retainer from the first portion at least partially into the second portion covers the gap therebetween.

8. The tray of claim 7, wherein the two elongated retainer side walls of the slidable device retainer further include one or more locking recesses for engagement with one or more locking protrusions along the elongated first retention side walls and second retention side walls to secure the slidable device retainer in at least one of the closed position and the open position.

9. The tray of claim 1, wherein the one or more device retainers are rotatable between the closed position and the open position.

10. The tray of claim 1, wherein the one or more retention pockets comprise a first rotational retention pocket and the one or more device retainers comprise a first rotatable device retainer configured to matingly fit and rotate within the first rotational retention pocket between the closed position and the open position.

11. The tray of claim 10, wherein the first rotatable device retainer includes a hub having a substantially circular hub wall that tapers as it extends towards a top flange portion of the hub, wherein the top flange portion is substantially planar.

12. The tray of claim 11, wherein the first rotational retention pocket includes a central ring having a device retainer center axis, a ring inner wall and a ridge portion, wherein the ring inner wall is substantially circular and tapers as it extends upward, and the hub is forcibly insertable into the central ring such that the circular hub wall is interlocked in rotatable abutment with the ring inner wall, and the top flange portion is received on and rotatable along the ridge portion.

13. The tray of claim 12, wherein the circular hub wall further includes one or more radially extending locking protrusions positioned to matingly engage locking recesses in the ring inner wall to secure the first rotatable device retainer in at least one of the closed position and open position.

14. The tray of claim 13, wherein the first rotational retention pocket comprises a first portion and a second portion separated by a gap, wherein the first portion includes the central ring, a first recessed ledge at least partially surrounding a first arced channel having a first channel bottom, and a first channel sidewall.

15. The tray of claim 14, wherein the second portion is rotationally aligned with the first portion and includes a second recessed ledge at least partially surrounding a second arced channel having a second channel bottom, and a second channel sidewall, and wherein rotation of the first rotatable device retainer about the device retainer center axis translates the first rotatable device retainer at least partially into the second portion and covers the gap between the first portion and the second portion.

16. The tray of claim 15, wherein the first channel sidewall and the second channel sidewall are evenly spaced from the central ring.

17. The tray of claim 16, wherein the first rotatable device retainer is substantially arc-shaped.

18. The tray of claim 1, wherein the tray body include a perimeter tray flange extending atop a plurality of tray walls, the plurality of tray walls interconnected along a tray bottom to form at least in part the tray body cavity.

19. The tray of claim 10, wherein the first rotatable device retainer includes a retainer side wall extending down from a perimeter lip.

20. The tray of claim 15, further comprising a second rotatable device retainer coupled to a second rotational retention pocket formed in the tray body wherein the second rotatable device retainer is rotatable from an open position to a closed position, and wherein the second rotatable device retainer remains coupled to the tray body regardless of its position.
